(19) United States
(12) Patent Application Publication
(10) Pub. No.: US 2021/0197401 A1
(43) Pub. Date: Jul. 1, 2021

(54) TOOL DETECTION SYSTEM

(71) Applicant: Auris Health, Inc., Redwood City, CA (US)

(72) Inventors: Jeffrey C. Weintraub, San Carlos, CA (US); Piyamate Wisanuvej, Redwood City, CA (US); Laurentiu Andrei Lita, San Jose, CA (US)

(21) Appl. No.: 17/131,581

(22) Filed: Dec. 22, 2020

Related U.S. Application Data
(60) Provisional application No. 62/956,002, filed on Dec. 31, 2019.

(51) Int. Cl. B25J 15/04; B25J 15/00; A61B 34/30; A61B 90/98

(52) U.S. Cl. CPC B25J 15/0483; B25J 15/0441; A61B 46/10; A61B 34/30; A61B 90/98; B25J 15/0019

(57) ABSTRACT

The systems and devices disclosed herein can permit a sterile adapter or manipulator interface to detect at least one attribute, parameter, and/or position of a tool attached to the instrument device manipulator. A sterile adapter can be coupled to the manipulator interface and include a magnet that is movable relative to the manipulator interface for permitting detection of the tool. Additionally, the present disclosure also relates to methods of preparing and using a medical robotic system.

### CROSS REFERENCE TO RELATED APPLICATIONS

This application claims the benefit of priority to U.S. Provisional Application No. 62/956,002, filed on Dec. 31, 2019, the disclosure of which is incorporated herein by reference in its entirety for all purposes.

### TECHNICAL FIELD

Systems, methods, and devices disclosed herein are related to medical robotic systems, and more particularly, to a tool detection system for a medical robot.

### BACKGROUND

Medical robots are increasingly being used to perform medical procedures such as endoscopy or laparoscopy because of the ability of these robots to manipulate tools in a way that humans traditionally could, with added advantages. For example, endoscopic or laparoscopic tools attached to medical robots can reduce the ergonomic load on the physician who can perform the procedures by manipulating the tools by commanding the robot from a comfortable position away from the site of the procedure. Additionally, because such tools can be constructed to enter and articulate into small spaces, the size of incision needed to perform the procedure can also be reduced or the tool may enter the patient through a natural orifice, thereby reducing recovery time.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates a medical robotic system, in accordance with some embodiments of the present disclosure.

FIG. 2 illustrates a command console for a medical robotic system, according to some embodiments.

FIG. 3 illustrates a perspective view of an instrument device manipulator (DM) for a medical robotic system, according to some embodiments.

FIG. 4 is a side view of the IDM, according to some embodiments.

FIGS. 5 and 6 illustrate perspective exploded views of an example tool coupled to the IDM of FIG. 3, according to some embodiments.

FIG. 7 illustrates a perspective view of a sterile adapter, in accordance with some embodiments.

FIG. 8 illustrates a cross-sectional view of the instrument shown in FIG. 7, taken along the line 8-8.

FIG. 9 illustrates a cross-sectional view of the instrument shown in FIG. 7, taken along the line 9-9 shown in FIG. 7.

### DETAILED DESCRIPTION

Disclosed herein is a medical robotic system including one or more robotic arms, each of which can be coupled to a robotic tool. The robotic tools, upon coupling to the robotic arms, can be manipulated by a user, such as a physician, using a console from an ergonomic position for performing the medical procedures. The advantages of such a medical robotic system include improvement in physician comfort, thereby enabling the physician to perform more complex tasks and for a longer period of time. As a result patient outcomes are improved. Additionally, such medical robotic systems enable minimally invasive procedures, thereby reducing recovery time.

According to some embodiments, a robotic arm of a medical robot is connected to an instrument device manipulator (IDM). The robotic arm can move the IDM to any position within a defined work space. Robotic tools such as a steerable catheter for endoscopic applications or any of a variety of laparoscopic tools can be connected to and manipulated by the IDM by activating control tools such as, for example, pull wires to steer a catheter or operate laparoscopic tools. Additionally, the IDM may be electrically and/or optically coupled to the tool to provide power, light, or control signals, and may receive data from the tool such as a video stream from a camera on the tool.

Because of the various operating mechanisms in the robotic arm and the IDM, it is generally difficult to completely sterilize the robotic arm and the DM. Moreover, the robotic arm and the IDM are capital equipment that cannot be disposed after a single use. Thus, while the robotic tools may be sterilized and disposed after use, a sterile barrier can be maintained between the IDM and the robotic tool when the robotic tool is coupled to the IDM. A surgical drape may separate the sterile space on the “tool-side” from the non-sterile space on the IDM side. However, various configurations of the IDM and the tool present challenges for draping the IDM and the robotic arm such as, for example, providing electrical, optical, and other connections between the draped IDM and the undraped tool.

According to some embodiments, a sterile adapter provides a sterile barrier between an IDM of a medical robotic system and a robotic tool. The sterile adapter may operatively couple to the IDM and to the robotic tool such that when the robotic tool is coupled to the sterile adapter, torque from the IDM is transferred to the robotic tool. The sterile adapter may additionally include a movable sensing target, such as a magnet movable relative to the IDM for permitting detection of the robotic tool that is coupled to the sterile adapter.

A robotic tool that can be coupled to a medical robot having a sterile adapter is also described. The robotic tool may include contact portion configured to cause a movement of a magnet of the sterile adapter relative to the IDM when the robotic tool is coupled to the sterile adapter. The amount of movement of the magnet relative to the IDM may facilitate identification of the tool. Additionally, different tools may be configured to depress a magnet of the sterile adapter to varying heights, or multiple magnets of the same or different heights may also be included to facilitate identification of different tools based on a depressed height of a magnet or a combination of magnets.

A medical robotic system including a sterile adapter and a robotic tool is also described. The medical robotic system may include an IDM including a sensor configured to sense a presence and position of a movable sensing target, such as a Hall effect sensor configured to sense a movable magnet positioned on the sterile adapter and determine a type of tool being coupled to the sterile adapter based on a signal generated by the sensor.

These and other features will be described in more detail below with reference to the embodiments illustrated in the figures, which are intended to illustrate certain example features and aspects of the technology described herein. The illustrated embodiments are not intended to be limiting, and those of skill in the art, upon consideration of this disclosure, will appreciate that various modifications can be made which are within the scope of this disclosure.

### A. Medical Robotic System

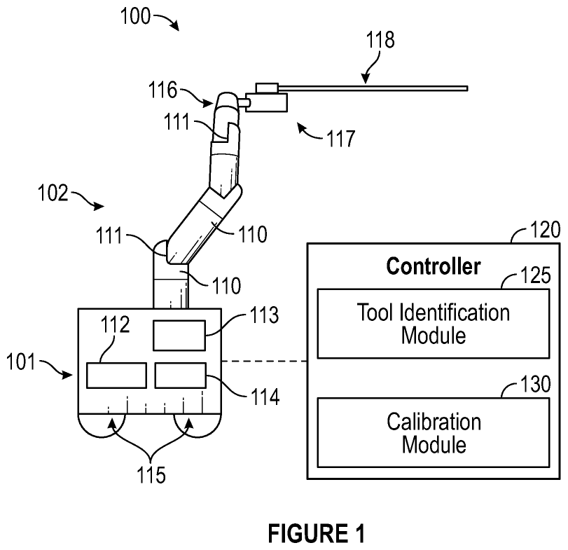

FIG. 1 illustrates a medical robotic system 100 in accordance with some embodiments of the present disclosure. The system 100 may include a base 101 coupled to one or more robotic arms 102. The base 101 can be positioned such that the robotic arm 102 has access to perform a medical procedure such as, for example, endoscopy, ureteroscopy, or laparoscopy, on a patient, while a user such as a physician may control the system 100 from an ergonomically comfortable site.

In some embodiments, the base 101 is optionally communicatively coupled to a command console located at the ergonomically comfortable site, using which the user can control the system 100. In some embodiments, the base 101 may be coupled to an operating table or bed for supporting the patient. Though not shown in FIG. 1 for purposes of clarity, the base 101 additionally or optionally includes subsystems such as control electronics, pneumatics, power sources, optical sources, and the like.

The base 101 may contain a source of power 112, pneumatic pressure 113, and control and sensor electronics 114—including components such as a central processing unit, data bus, control circuitry, and memory—and related actuators such as motors to move the robotic arm 102. The electronics 114 in the base 101 may also process and transmit control signals communicated from the command console.

In some embodiments, the base 101 may include wheels 115 to transport the system 100. Mobility of the system 100 advantageously helps accommodate space constraints in an operating room as well as facilitates appropriate positioning and movement of medical equipment. Further, the mobility allows the robotic arms 102 to be configured such that the robotic arms 102 do not interfere with the patient, physician, anesthesiologist, or any other equipment. During procedures, a user may control the robotic arms 102 using control devices such as the command console.

In some embodiments, the robotic arm 102 includes multiple arm segments 110 coupled at joints 111, which provide the robotic arm 102 multiple degrees of freedom, e.g., seven degrees of freedom corresponding to seven arm segments. In some embodiments, the robotic arm 102 includes set up joints that use a combination of brakes and counter-balances to maintain a position of the robotic arm 102. The counter-balances may include gas springs or coil springs. The brakes, e.g., fail safe brakes, may include mechanical and/or electrical components. In some embodiments, the robotic arms 102 may be gravity-assisted passive support type robotic arms.

Each robotic arm 102 may be coupled to an IDM 117 using a mechanism changer interface (MCI) 116. The IDM 117 can be removed and replaced with a different type of IDM, for example, a first type of IDM may manipulate an endoscope, while a second type of IDM may manipulate a laparoscope. In some embodiments, the IDM 117 may manipulate medical tools such as, for example, an endoscope 118 using manipulation methods including direct drive, harmonic drive, geared drives, belts and pulleys, magnetic drives, or any combination thereof.

In some embodiments, the MCI 116 may include connectors to transfer pneumatic pressure, electrical power, electrical signals, and optical signals from the robotic arm 102 to the IDM 117. The MCI 116 can be a set screw or base plate connector. The MCI 116 can be interchangeable based on the type of IDM 117 and can be customized for a certain type of surgical procedure.

The endoscope 118 is a tubular and flexible instrument that is inserted into the anatomy of a patient to capture images of the anatomy (e.g., body tissue). In some embodiments, the endoscope 118 may include one or more imaging devices (e.g., cameras or sensors) that capture the images. The imaging devices may include one or more optical components such as an optical fiber, fiber array, or lens. The optical components move along with the tip of the endoscope 118 such that movement of the tip of the endoscope 118 results in changes to the images captured by the imaging devices. It will be appreciated that while an endoscope is used as the primary example throughout, it is understood that the surgical robotic system 100 may be used with a variety of surgical instruments.

In some embodiments, robotic arms 102 of the system 100, and more particularly, the IDMs 117 of the corresponding robotic arms 102, manipulate the endoscope 118 using elongate movement members. The elongate movement members may include pull-wires, also referred to as pull or push wires, cables, fibers, or flexible shafts. For example, the robotic arms 102 actuate multiple pull-wires coupled to the endoscope 118 to deflect the tip of the endoscope 118. The pull-wires may include both metallic and non-metallic materials such as stainless steel, Kevlar, tungsten, carbon fiber, and the like. In some embodiments, the endoscope 118 may exhibit nonlinear behavior in response to forces applied by the elongate movement members. The nonlinear behavior may be based on stiffness and compressibility of the endoscope 118, as well as variability in slack or stiffness between different elongate movement members.

FIG. 1 also shows that the system 100 may include a controller 120, for example, a computer processor. In some embodiments, the controller 120 may include a tool identification module 125 and a calibration module 130. The tool identification module 125 can identify the robotic tool 118 being coupled to the IDM 117 and cause the system 100 to produce a confirmation signal, e.g., an audible and/or visual confirmation, that the tool 118 being connected to the IDM 117 is the intended tool. Conversely, the system 100 may produce a warning signal, e.g., an audible and/or a visual alert, that an incorrect tool has been connected to the IDM 117. Such confirmation and/or alert enables the user such as a physician to non-visually couple the correct tool 118 during a procedure and provides a safety mechanism if it becomes necessary to change the tool 118 during the procedure.

Once the tool 118 coupled to the IDM 117 is identified, the calibration module 130 can characterize the nonlinear behavior of the identified tool 118 coupled to the IDM 117. In some embodiments, the characterization of the nonlinear behavior may be performed using a model with piecewise linear responses along with parameters such as, for example, slopes, hystereses, and dead zone values. The system 100 can more accurately control the robotic tool 118 by determining accurate values of the parameters. In some embodiments, some or all functionality of the controller 120 is performed outside the system 100, for example, on another computer system or server communicatively coupled to the system 100.

### B. Command Console

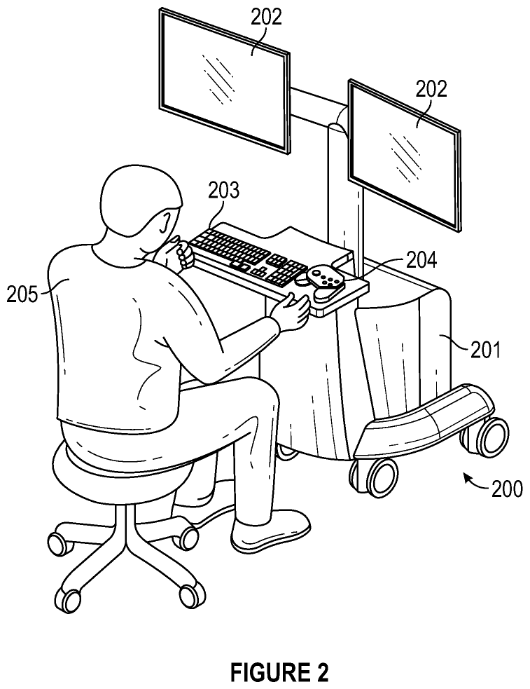

FIG. 2 illustrates a command console 200 for a medical robotic system 100 according to some embodiments of the present disclosure. The command console 200 includes a console base 201, display modules 202, e.g., monitors, and control modules, e.g., a keyboard 203 and joystick 204. In some embodiments, one or more of the command module 200 functionality may be integrated into a base 101 of the surgical robotic system 100 or another system communicatively coupled to the surgical robotic system 100. A user 205, e.g., a physician, remotely controls the system 100 from an ergonomic position using the command console 200.

The console base 201 may include a central processing unit, a memory unit, a data bus, and associated data communication ports that are responsible for interpreting and processing signals such as camera imagery and tracking sensor data, e.g., from the tool 118 shown in FIG. 1. In some embodiments, both the console base 201 and the base 101 perform signal processing for load-balancing. The console base 201 may also process commands and instructions provided by the user 205 through the control modules 203 and 204. In addition to the keyboard 203 and joystick 204 shown in FIG. 2, the control modules may include other devices, for example, computer mice, track pads, trackballs, control pads, video game controllers, and sensors (e.g., motion sensors or cameras) that capture hand gestures and finger gestures.

The display modules 202 may include electronic monitors, virtual reality viewing devices, e.g., goggles or glasses, and/or other means of display devices. In some embodiments, the display modules 202 are integrated with the control modules, for example, as a tablet device with a touchscreen. Further, the user 205 can both view data and input commands to the system 100 using the integrated display modules 202 and control modules, in some embodiments.

### C. Instrument Device Manipulator

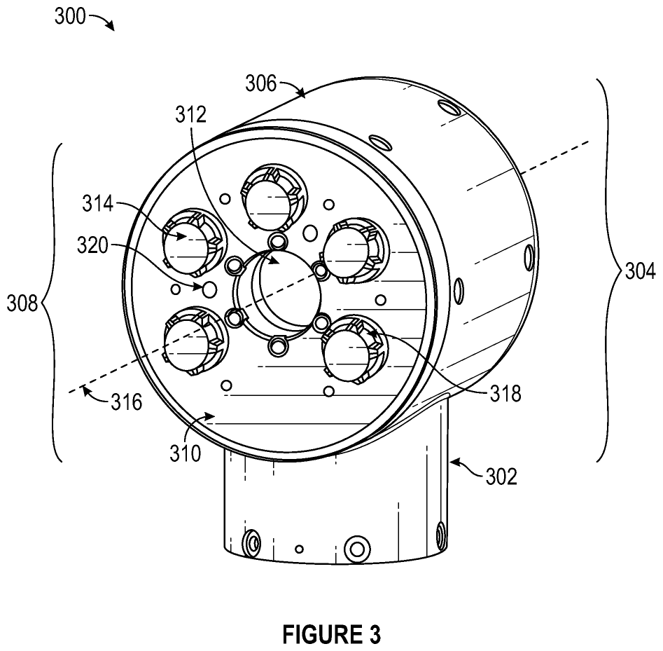

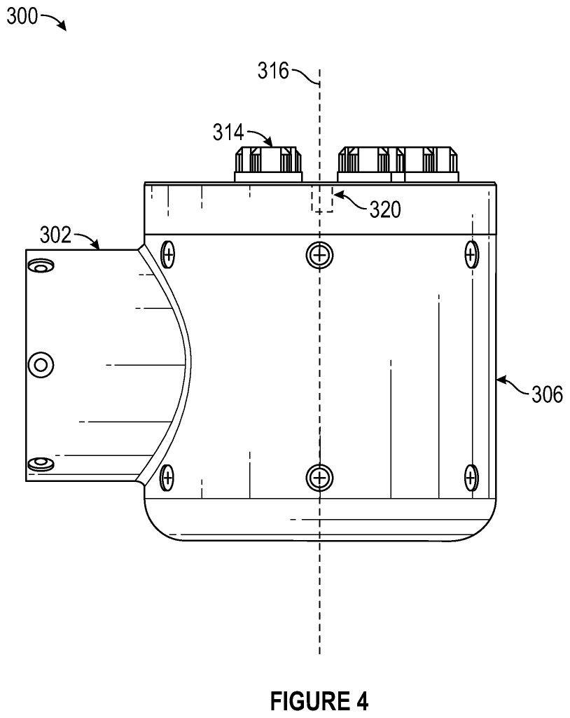

FIG. 3 illustrates a perspective view of an IDM 300 for a medical robotic system, and FIG. 4 is a side view of the IDM 300, according to some embodiments of the present disclosure. The IDM 300 is configured to attach a robotic tool such as the tool 118 to a robotic arm. In some embodiments, the IDM 300 is configured to attach to the robotic tool in a manner that allows the tool to be continuously rotated or “rolled” about an axis of the tool. The IDM 300 includes a base 302 and a tool holder assembly 304. The tool holder assembly 304 further includes an outer housing 306, a tool holder 308, an attachment interface 310, a plurality of torque couplers 314, and optionally a passage 312. The IDM 300 may be used with a variety of medical tools (not shown in FIG. 3), which may include a housing and an elongated body, and which may be used for procedures such as laparoscopy, endoscopy, or ureteroscopy. The medical tools may have articulable components, such as deflectable distal tips for steering, actuatable wrists (e.g., for grasping, cutting, etc.), or other manipulatable components for interacting with target sites within a patient (e.g., energy delivery devices, basketing tools, extendable needles, etc.). Thus, the medical tools may have different end-effectors suitable for the corresponding procedure.

The base 302 removably or fixedly mounts the IDM 300 to a robotic arm of the system. In the embodiment of FIG. 3, the base 302 is fixedly attached to the outer housing 306 of the surgical tool holder assembly 304. In some embodiments, the base 302 may be structured to include a platform which is adapted to rotatably receive the tool holder 308 on the face opposite from the attachment interface 310. The platform may optionally include a passage aligned with the passage 312 to receive the elongated body of the tool and, in some embodiments, an additional elongated body of a second surgical tool mounted coaxially with the first surgical tool.

The tool holder assembly 304 is configured to secure a tool to the IDM 300 and can be configured to rotate the tool relative to the base 302. In some embodiments, mechanical and electrical connections may be provided from the surgical arm to the base 302 and then to the tool holder assembly 304 to rotate the tool holder 308 relative to the outer housing 306. The connections facilitate manipulation and/or delivery power and/or signals from the robotic arm to the tool holder 308 and ultimately to the tool. Signals may include signals for pneumatic pressure, electrical power, electrical signals, and/or optical signals.

In some embodiments, the outer housing 306 provides support for the surgical tool holder assembly 304 with respect to the base 302. The outer housing 306 may be fixedly attached to the base 302 such that it remains stationary relative to the base 302, while allowing the tool holder 308 to rotate freely relative to the outer housing 306. In the embodiment of FIG. 3, the outer housing 306 is cylindrical in shape and fully circumscribes the surgical tool holder 308. The outer housing 306 may be composed of rigid materials (e.g., metals or hard plastics). It will be appreciated that the shape of the housing may vary.

The tool holder 308 functions to secure a tool to the IDM 300 via the attachment interface 310. The tool holder 308 is capable of rotating independent of the outer housing 306. The tool holder 308 rotates about a rotational axis 316, which co-axially aligns with the elongated body of a tool such that the tool rotates with the tool holder 308.

The attachment interface 310 is a face of the tool holder 308 that attaches to the tool. The attachment interface 310 may include a first portion of an attachment mechanism that reciprocally mates with a second portion of the attachment mechanism located on the tool. In some embodiments, the attachment interface 310 includes a plurality of torque couplers 314 that protrude outwards from the attachment interface 310 and engage with respective instrument inputs on the tool. In some embodiments, a surgical drape, coupled to a sterile adapter, may be used to create a sterile boundary between the IDM 300 and the tool. In these embodiments, the sterile adapter may be positioned between the attachment interface 310 and the tool when the tool is secured to the IDM 300 such that the surgical drape separates the tool and the patient from the IDM 300 and the robotic system.

In some embodiments, the plurality of torque couplers 314 are configured to engage and drive the components of the tool when the tool is secured to the tool holder 308. Each torque coupler 314 is inserted into a respective instrument input located on the tool. The plurality of torque couplers 314 may also serve to maintain rotational alignment between the tool and the tool holder 308.

As illustrated in FIG. 3, each torque coupler 314 is shaped as a cylindrical protrusion that protrudes outwards from the attachment interface 310. Notches 318 may be arranged along the outer surface area of the cylindrical protrusion. In some embodiments, the arrangement of the notches 318 creates a spline interface. The instrument inputs on the tool are configured to have a complementary geometry to the torque couplers 314.

For example, while not shown in FIG. 3, the instrument inputs of the tool may be cylindrical in shape and have a plurality of ridges that reciprocally mate with the plurality of notches 318 on each torque coupler 314 and thus impart a torque on the notches 318. In some embodiments, the top face of the cylindrical protrusion may include the plurality of notches 318 configured to mate with a plurality of ridges in respective instrument inputs. In this configuration, each torque coupler 314 fully engages with its respective instrument input.

Each torque coupler 314 may be driven by a respective actuator that causes the torque coupler to rotate in either direction. Thus, once engaged with an instrument input, each torque coupler 314 is capable of transmitting power to tighten or loosen pull-wires within a tool, thereby manipulating a tool's end-effectors. In the embodiment of FIG. 3, the IDM 300 includes five torque couplers 314, but the number may vary in other embodiments depending, for example, on the desired number of degrees of freedom for a tool's end-effectors or the construction of internal mechanisms of the tool.

In some embodiments, a surgical drape, coupled to a sterile adapter, may be used to create a sterile boundary between the IDM 300 and the surgical tool. In these embodiments, the sterile adapter may be positioned between the attachment interface 310 and the tool when the surgical tool is secured to the IDM 300, and the sterile adapter may be configured to transmit power from each torque coupler 314 to the respective instrument input.

The IDM 300 can optionally include a sensor that can detect attributes, parameters, and/or position of a tool attached to the IDM 300.

For example, the IDM 300 can comprise one or more Hall effect sensors 320 for sensing a magnetic field associated with a magnet disposed on the tool or a change in the magnetic field associated with a magnet movably disposed on the sterile adapter. Although examples are described herein with respect to Hall effect sensor 320 in an IDM or movable magnets in a sterile adapter, it is contemplated that any of a variety of sensors in the IDM, or movable targets in an intermediate device between the tool and IDM, may be used to detect presence or attributes of a tool coupled to the IDM. For example, in some embodiments, capacitive sensors, inductive proximity sensors, or other types of electronic sensors may be utilized in the IDM to detect a movable sensor target in the intermediate device. Additionally, conductive targets (which need not necessarily be magnetic),or other types of movable targets detectable by an electronic sensor, may be utilized in the intermediate device to detect the tool.

A typical Hall effect sensor outputs a voltage based on a magnitude and direction of a magnet field at the Hall effect sensor. Thus, the Hall effect sensor 320 may be used to detect presence of or change in a magnetic field. For example, in some embodiments, the Hall effect sensor 320 may sense the magnitude and direction of the magnetic field associated with a magnet disposed on the tool to output a certain voltage based on the sensed magnetic field.

In some embodiments, the change in the magnetic field associated with a magnet disposed on the sterile adapter may be sensed using the Hall effect sensor 320 by measuring a difference in voltage output by the Hall effect sensor 320 before and after a certain event, e.g., coupling of the tool to the sterile adapter. In some embodiments, such change in magnetic field may be a result of a change in a position of the magnet disposed on the sterile adapter when the tool is coupled to the sterile adapter. In some embodiments, the change in magnetic field associated with the magnet disposed on the sterile adapter may be caused by a magnet disposed on the tool which modulates the magnetic field of associated with the magnet disposed on the sterile adapter when the tool is coupled to the sterile adapter. In either instance, the Hall effect sensor 320 generates a signal (e.g., a change in output voltage) based on the sensed magnetic field or the sensed change in magnetic field.

The signal produced by the Hall effect sensor 320 may provide information about the type of the tool being coupled to the IDM 300, e.g., based on different magnetic field strengths produced by different tools, or based on the different changes in magnetic fields produced by the sterile adapter caused by different tools being coupled to the sterile adapter.

In some embodiments, the IDM can comprise two or more Hall effect sensors 320, which can correspond, for example, to two or more magnets on the tool or the sterile adapter. Magnetic fields or changes in magnetic fields sensed by the two or more Hall effect sensors 320 may be used to identify the type of tool being coupled to the IDM 300.

In some embodiments, the IDM can have a first Hall effect sensor and a second Hall effect sensor. First and second tools can be coupled to the IDM that produce unique magnetic fields detectable by the first and second Hall effect sensors of the IDM.

For example, the first tool can have two magnets that may produce magnetic fields B1 and B2 respectively at the first and second Hall effect sensors 320. Further, the second tool can have two magnets that may produce magnetic fields B3 and B4 respectively at the first and second Hall effect sensors 320.

Likewise, in some embodiments, a first tool having two magnets may change the magnetic fields by ΔB1 and ΔB2 respectively at the first and second Hall effect sensors 320. A second tool having two magnets may change the magnetic fields by ΔB3 and ΔB4 respectively at the first and second Hall effect sensors 320. By determining the magnetic fields (or changes in the magnetic fields) at the first and second Hall effect sensors 320, the system can determine whether the first tool or the second tool is coupled to the IDM.

In some embodiments, the IDM 300 can have three Hall effect sensors 320 corresponding to three magnets on the sterile adapter. The three magnets in the sterile adapter may define a plane that can be determined based on the relative field strength sensed by the three corresponding Hall effect sensors 320. When only the sterile adapter is properly attached (i.e., no tool is attached), the plane determined by the system can be stored within the system. Thus, the system can recognize when the sterile adapter is properly latched on the IDM 300 based on recognition of the plane formed by the three magnets.

Using a similar mechanism, the system may detect when a tool is properly latched on to a properly latched sterile adapter. For example, one or more relative field strengths detected by the Hall effect sensors 320 corresponding to one or more of the magnets may be used to determine the type of tool coupled to the IDM 300. The different planes formed by the three magnets corresponding to different tools can be stored in the system. The system may first recognize which tool is attached, and then determine whether that tool is properly attached by comparing the plane formed by the tool upon attachment to the plane corresponding to that tool stored in the system.

Advantageously then, detection of the plane corresponding to a properly latched tool may be helpful in preventing the system from driving a non-latched or partially-latched tool. Improper torque transmission to the tool or accidental detachment of the tool thus be prevented, thereby improving patient safety.

In some embodiments, the Hall effect sensor 320 may be operatively coupled to a controller of the IDM 300. The controller, upon receiving the signal from the Hall effect sensor 320, may determine whether a tool is being coupled to the IDM 300, and/or identify the tool being coupled to the IDM 300, and/or identify whether the tool is properly latched. The controller may then produce or cause the system to produce a confirmation or an alert signal. The confirmation or the alert signals may be audio, visual, audiovisual, and/or haptic, in some embodiments. The confirmation or alert signals serve to provide the user with a feedback about whether a proper tool has been coupled to the IDM or the sterile adapter.

### D. Sterile Adapter

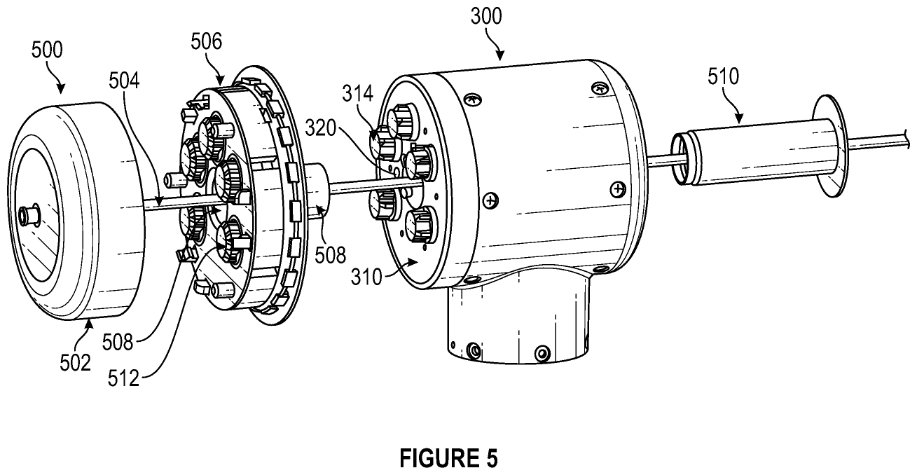

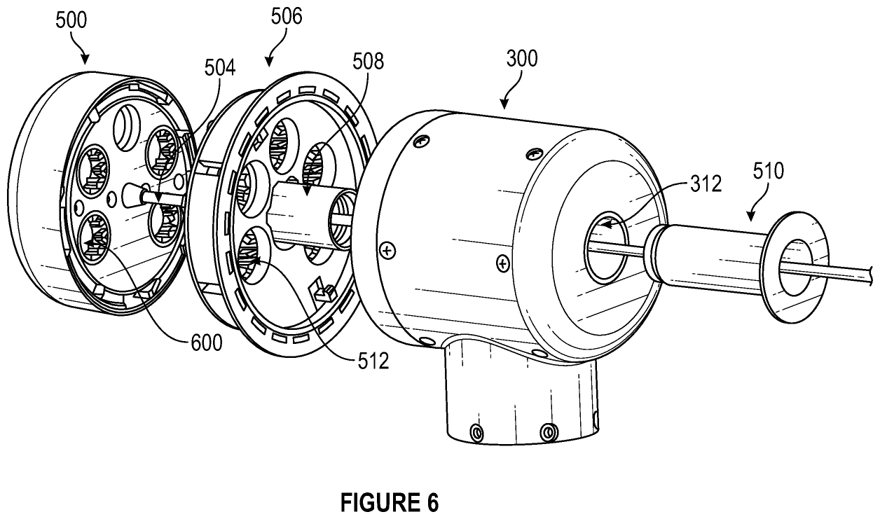

FIGS. 5 and 6 illustrate perspective exploded views of an example tool 500 coupled to the IDM 300 of FIG. 3, according to some embodiments of the present disclosure. The tool 500 includes a housing 502, an elongated body 504, and a plurality of instrument inputs 600. As previously described, the elongated body 504 may be a laparoscope, an endoscope, or other surgical instrument having end-effectors. As illustrated, the plurality of torque couplers 314 protrude outwards from the attachment interface 310 to engage with the instrument inputs 600 of the surgical tool. The structure of the instrument inputs 600 can be seen in FIG. 6, wherein the instrument inputs 600 have corresponding geometry to the torque couplers 314 to ensure secure surgical tool engagement.

During a medical procedure, in order to maintain a sterile boundary between the IDM 300 and the tool 500, a sterile adapter 506 may be coupled to the IDM 300 intermediate the tool 500 and the IDM 300. As further described herein, the sterile adapter 506 may include features, such as a movable magnet or other sensor target, to detect presence or attributes of a tool upon coupling the tool to the sterile adapter 506.

While examples are described herein with respect to a sterile adapter, it is contemplated that any features disclosed herein with respect to the sterile adapter may be used in any other adapter or other intermediate device intermediate the IDM and the tool. For example, in some embodiments, a non-sterile adapter may be mounted to the IDM and include movable magnets or other movable sensor targets to facilitated detection of a tool mounted to the non-sterile adapter.

While not shown in FIGS. 5 and 6, in some embodiments, a sterile sheet may be connected to the sterile adapter 506 so as to drape around the IDM 300 to create the sterile boundary. The sterile adapter 506 can create a sterile interface between the IDM 300 and the tool 500 when secured to the IDM 300. In the embodiment of FIGS. 5 and 6, the sterile adapter 506 has a disk-like geometry that covers the attachment interface 310 of the IDM 300. The sterile adapter 506 includes a central hole configured to receive the elongated body 504 of the surgical tool 500. In this configuration, the sterile adapter 506 is positioned between the attachment interface 310 and the tool 500 when the tool 500 is secured to the IDM 300, creating the sterile boundary between the tool 500 and the IDM 300 and allowing the elongated body 504 to pass through the passage 312.

In some embodiments, the sterile adapter 506 may be capable of rotating with the tool holder 308, transmitting the rotational torque from the plurality of torque couplers 314 to the tool 500, passing electrical signals between the IDM 300 and the tool 500, or some combination thereof.

The sterile adapter 506 can include a first protrusion 508 and a second protrusion 510 to maintain a sterile boundary between the tool and the IDM within the passage 312. The first protrusion 508 and the second protrusion 510 are configured to pass through the passage 312 of the IDM 300 and mate with each other inside the passage 312. Each protrusion 508, 510 is structured to allow the elongated body 504 to pass through the protrusion and thus the passage 312. The connection of the first protrusion 508 and the second protrusion 510 creates the sterile boundary between the IDM 300 and the outside environment (i.e., an operating room).

It will be understood that while the Figures illustrate a certain geometry and configuration of the sterile adapter 506 and the IDM 300, other configurations and geometries are contemplated within the scope of the present disclosure. For example, the sterile adapter may have a square or a rectangular shape depending on the shape of the IDM and/or the tool. In some embodiments, the elongate body 504 of the tool 500 may extend transversely to the central axis 316 of the IDM 300, and thus, need not pass through the sterile adapter 506 or the IDM 300. In such configurations, the sterile adapter 506 need not include a central hole or the first protrusion 508.

In the embodiment illustrated in FIGS. 5 and 6, the sterile adapter 506 further includes a plurality of couplers or drive members 512. A first side of a coupler 512 is configured to engage with a respective torque coupler 314 while a second side of a coupler 512 is configured to engage with a respective instrument input 600. Similar to the structure of the plurality of torque couplers 314, each coupler 512 is structured as a cylindrical protrusion including a plurality of notches.

Optionally, each side of the coupler 512 can have complementary geometry to fully engage with the respective torque coupler 314 and the respective instrument input 600. Each coupler 512 can be configured to rotate in a clockwise or counter-clockwise direction with the respective torque coupler 314. This configuration allows each coupler 512 to transfer torque and rotary motion from the plurality of torque couplers 314 of the IDM 300 to the plurality of instrument inputs 600 of the tool 500, and thus control the end-effectors of the tool 500.

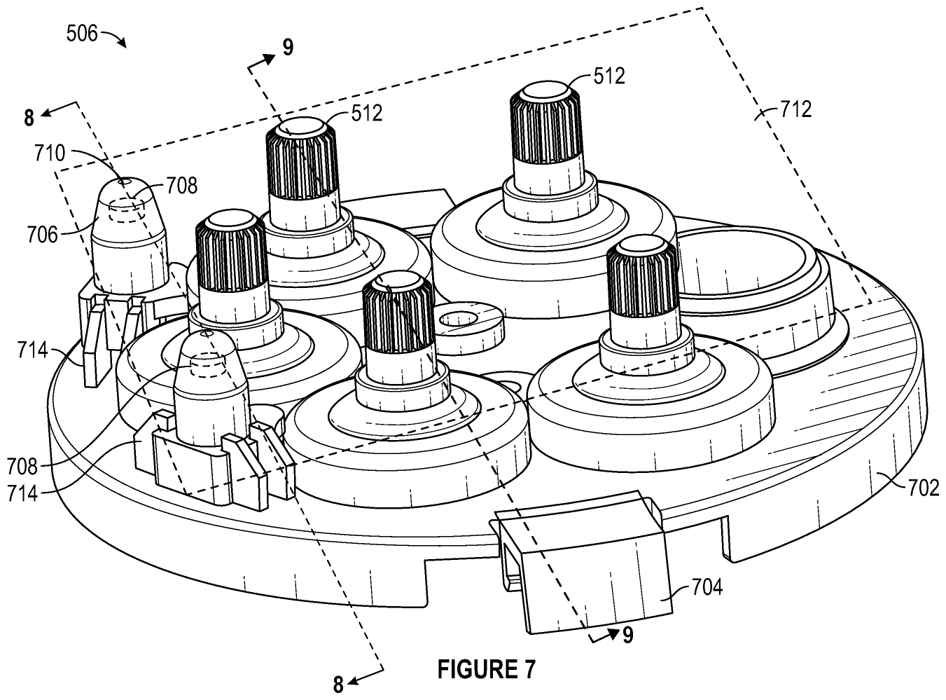

FIG. 7 illustrates a perspective view of a bottom half of a sterile adapter 506 in accordance with some embodiments of the present disclosure. In the embodiment shown in FIG. 7, the sterile adapter 506 includes couplers 512. The sterile adapter also comprises a lower housing 702. The lower housing 702 can comprise a latch 704 for attaching the sterile adapter 506 to an IDM of a medical robotic system.

As discussed herein, a medical instrument (e.g., tool 500, shown in FIG. 6) can be coupled to the medical robotic system by coupling the tool to the sterile adapter 506. When the tool is coupled to the sterile adapter 506, the couplers 512 transfer torque from the IDM to the input drives. Thus, couplers 512 have a shape on the IDM side corresponding to the shape of the drive outputs, such as the torque couplers 314, of the IDM. On the tool side the couplers 512 have a shape (shown in FIG. 7) corresponding to the shape of the input drives of the tool.

In some embodiments, the sterile adapter 506 can include a magnet 708 coupled to a plunger component 706. The magnet 708 may be movable relative to the IDM to which the sterile adapter 506 is being coupled. In some embodiments, the plunger component 706 may be coupled to the lower housing 702. In some embodiments, magnet 708 may be coupled on an underside of a plunger tip portion 710 of the plunger component 706, thereby providing a default position for the magnet 708. The default position may provide a baseline magnetic field to be sensed by a Hall effect sensor of the IDM. In some embodiments, the plunger tip portion 710, in a default position, extends a certain distance from a tool interface plane 712 in a direction transverse to the lower housing 702. The tool interface plane 712 may be a defined as a plane at which the tool couples with the sterile adapter 506.

In some embodiments, the plunger component 706 is formed of a flexible material such as, for example, rubber or silicone. The plunger component 706 may allow the magnet 708 to move away from its default position. For example, in some embodiments, the plunger component 706 may allow the magnet to move towards the lower housing 702, relative to the IDM along an axis parallel to the central axis of the sterile adapter 506. The plunger component 706 may be positioned on the lower housing 702 such that a Hall effect sensor of the IDM can sense a change in magnetic field generated by the magnet 708.

In some embodiments, the change in the magnetic field generated by the magnet 708 may be caused by the movement of the magnet 708 when a plunger contact portion correspondingly positioned on the tool moves the plunger tip portion 710 away from its default position once the tool is coupled to the sterile adapter 506. For example, in some embodiments, the plunger tip portion 710, and thereby the magnet 708, may be moved by a distance in a range from about 0.5 mm to about 5 mm, e.g., about 0.5 mm, about 1 mm, about 1.5 mm, about 2 mm, about 2.5 mm, about 3 mm, about 3.5 mm, about 4 mm, about 4.5 mm, about 5 mm, or any other distance between any two of these values.

In some embodiments, the plunger component 706 may comprise a rigid body coupled to a spring (not shown), e.g., a coil spring or a leaf spring, which biases the plunger component 706 to a default position relative to the lower housing 702. The spring may be compressed when the plunger contact portion of the tool pushes against the plunger tip portion 710, causing the plunger component 706, and thereby the magnet 708 to move relative to the DM. In some embodiments, the plunger component 706 may be made of a flexible material such as, for example, rubber or silicone, that allows the plunger component 706 to get compressed when the plunger contact portion of the tool presses against the plunger tip portion 710.

While a plunger component with a generally cylindrical body is shown in FIG. 7, it will be appreciated that the structure of the plunger component provided on the sterile adapter may vary. For example, the plunger component may have a conical or a pyramidal structure in some embodiments. In some embodiments, the plunger component may have a polygonal cross-section.

Further, in some embodiments, the plunger component may comprise a cantilevered or leaf spring member. The cantilevered member can extend along or generally within the tool interface plane 712 of the housing (e.g., as defined by the flat surface of the lower housing 702 from which the couplers 512 and plunger components 706 extend) in a default position, with the magnet being disposed at the untethered or free end portion of the cantilevered member. The plunger contact portion of the tool, upon coupling of the tool, may contact the untethered end portion (e.g., a cantilevered contact head) of the cantilever member, causing displacement of the cantilevered contact head and thereby moving the magnet from its default position. In turn, the displacement of the cantilevered contact head will permit the Hall effect sensor to detect movement of the magnet.

In some embodiments, the plunger component 706 may comprise an alignment feature 714. The alignment feature 714 can comprise one or more protrusions, indentations, flanges, or grooves that can interact with one or more corresponding features of the lower housing 702 to facilitate or control movement of the plunger component 706. For example, the alignment feature 714 can tend to restrict motion of the plunger component 706 in certain directions while permitting the plunger component 706 to translate in a direction normal relative to the tool interface plane 712.

Further, the alignment feature 714 can align the plunger component 706 with the Hall effect sensor of the IDM and the plunger contact portion of the tool. In some embodiments, the alignment feature 714 can restrict movement of the plunger component 706, and thereby the magnet 708, to an axial direction that is oriented parallel to the central axis of the sterile adapter 506.

In some embodiments, the sterile adapter 506 may include more than one plunger component 706. Further, each plunger component 706 can include a magnet 708. For example, FIG. 7 illustrates a sterile adapter 506 having two plunger components 706. However, more than two plunger components 706 can be incorporated into the sterile adapter 506, in accordance with some embodiments.

In some embodiments, when coupled to the sterile adapter 506, each one of the different tools can contact the plunger tip portion 710 and cause displacement of the plunger component 706 relative to the lower housing 702 by a given distance. Each tool can cause a distinct amount of displacement of a given plunger component 706.

In this manner, the magnet 708 associated with a respective plunger component 706 can move to one of a plurality of different positions relative to the Hall effect sensor. The Hall effect sensor may generate different signals depending on such different positions which result in different amounts of change in the magnetic field associated with the magnet 708. Thus, the IDM or the controller associated with the IDM can determine whether the sterile adapter 506 is properly latched to the IDM based on the different signals generated by the Hall effect sensor. In some embodiments, the IDM or the controller associated with the IDM can determine the type of tool being coupled to the sterile adapter 506 as well as whether that type of tool is properly latched onto the sterile adapter 506 based on the different signals generated by the Hall effect sensor.

Thus, the moveable magnet 708 in the sterile adapter 506 can assist the robotic system in determining whether a tool has been coupled to the system, whether the tool was correctly coupled to the system, and other attributes of the tool, such as which tool has been coupled to the system for a given procedure being conducted by the user. For example, the system may also be able to determine if a given tool is properly coupled to the sterile adapter 506 by comparing the signal generated by the Hall effect sensor with a tool is attached to an ideal value associated with the given tool.

### E. Instruments for Performing Medical Procedures

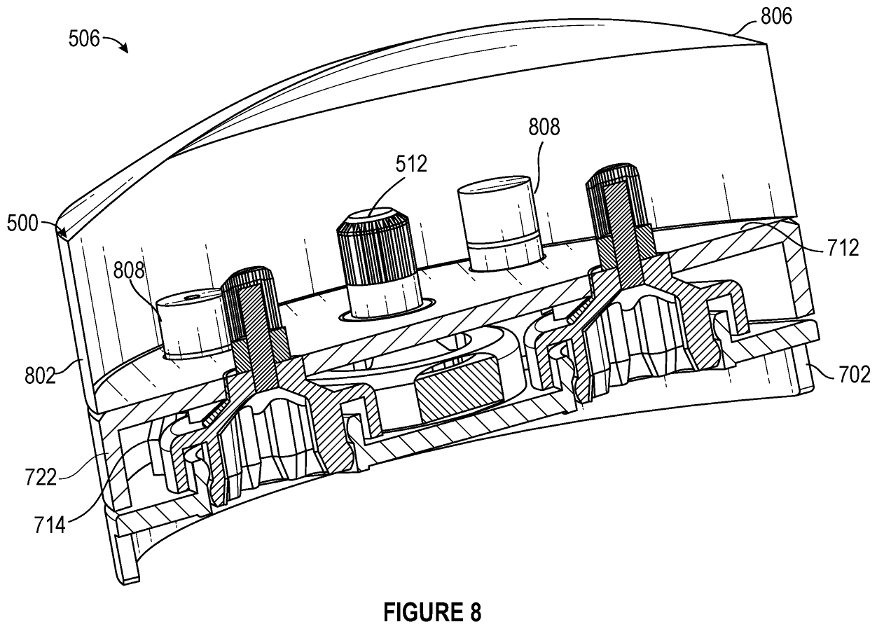

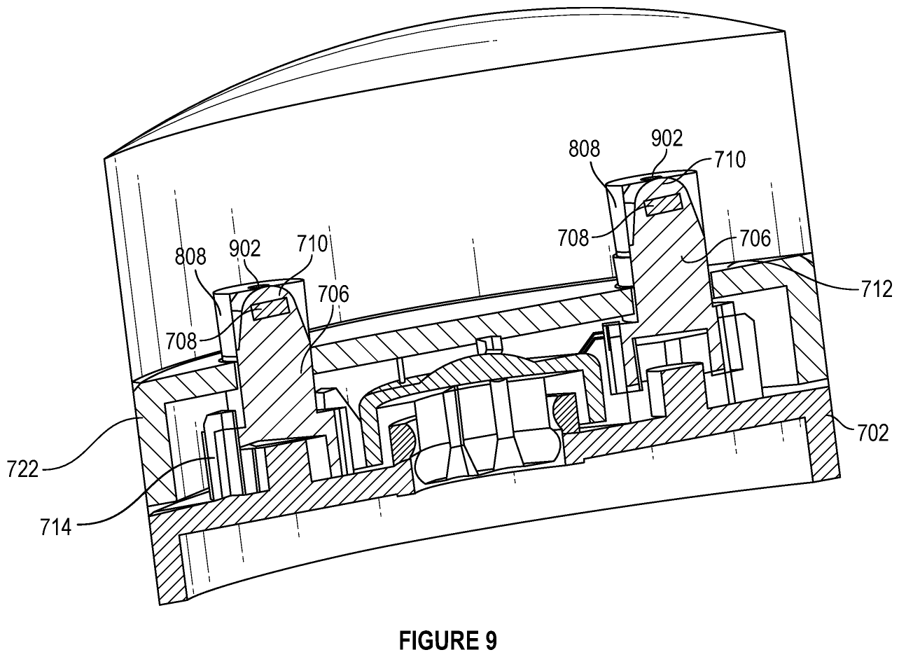

FIGS. 8 and 9 illustrate cross-sectional view of the instrument shown in FIG. 7. FIG. 8 illustrates a cross-section view of the instrument for performing a medical procedure (referred to herein as the “tool”) coupled to a sterile adapter, taken along the line 8-8 shown in FIG. 7. Line 8-8 cuts across two of the couplers 512. FIG. 9 also illustrates a cross-section view of an instrument for performing a medical procedure coupled to a sterile adapter, taken along the line 9-9 shown in FIG. 7. Line 9-9 cuts across two of the plunger components 706.

The tool 500 may include a tool interface 802 defining a tool interface plane 712. The tool interface 802 couples the tool 500 to a sterile adapter disclosed herein, e.g., the sterile adapter 506. The tool 500 may further include a plunger contact portion 808. It should be noted that in FIG. 8, the structure of the tool input drivers replaced by corresponding apertures so as to better illustrate the drive couplers 512 of the sterile adapter past the tool interface 802. FIGS. 8 and 9 further illustrate an upper housing 722 of the sterile adapter to which the tool 500 latches.

In some embodiments, the plunger contact portion 808 has a shape corresponding to a plunger component. For example, the plunger contact portion 808 may be shaped corresponding to the plunger component 706, of an adapter, such as the sterile adapter 506. The plunger contact portion 808 may be positioned such that when the tool 500 is coupled to the sterile adapter, the plunger contact portion 808 moves a plunger tip portion of the plunger component.

For example, the plunger contact portion 808 may be positioned to move, when the tool interface 802 couples with the upper housing 722, the plunger tip portion 710 of the plunger component 706. In the embodiment shown in FIG. 8, the plunger contact portion 808 is formed as a cavity in the tool housing 806 in which the plunger component can be received to a certain distance as the tool is coupled to the sterile adapter.

In some embodiments, the plunger contact portion 808 may be constructed and structured to move the plunger tip portion by a particular distance based on the type of tool. The tool may be identified based on the amount of movement of the plunger tip portion.

For example, the plunger contact portion 808 for a basketing tool may be constructed to move the plunger tip portion by a first distance X1. The plunger contact portion 808 for an ureteroscope may be constructed to move the plunger tip portion by a second distance X2. Thus, upon coupling of the tool, the plunger tip portion of the sterile adapter, and thereby a magnet coupled to the plunger tip portion, may move a distance X1 or X2 depending on which tool is being coupled. The movement of the magnet by different distances results in different changes in the magnetic field associated with the magnet being sensed by the Hall effect sensor of the IDM. The different changes in the magnetic field generate different signals. The different signals can be used to determine the type of tool being coupled.

In some embodiments, the tool 500 may include multiple plunger contact portions 808 which engage with corresponding plunger components of a sterile adapter. For example, in the embodiment shown in FIG. 9, the tool has two plunger contact portions 808, each having a cavity ceiling 902. Different cavity ceilings 902 may move the corresponding plunger tip portions 710 by different distances that can be designed to be specific to the type of the tool.

For example, in some embodiments, the IDM may include 3 Hall effect sensors 320 at positions A, B and C. A first tool may include two plunger contact portions corresponding only to positions A and B, such that the corresponding magnets of the sterile adapter are moved by distances X1 and X2 with respect to the Hall effect sensor 320 at positions A and B. A second tool may include two plunger contact portions corresponding only to positions A and B, but constructed to move the corresponding magnets of the sterile adapter by distances Y1 and Y2 (different from X1 and X2). A third tool may include two plunger contact portions corresponding only to positions A and B, but constructed to move the corresponding magnets of the sterile adapter by distances X1 and Y2. A fourth tool may include two plunger contact portions corresponding to positions A and C, constructed to move the corresponding magnets of the sterile adapter by distances X1 and X3. A fifth tool may include two plunger contact portions corresponding to positions A and C, constructed to move the corresponding magnets of the sterile adapter by distances X1 and Y3.

Thus, various combinations of positions at which the plunger contact portions are disposed and the distances by which the plunger contact portions move corresponding magnets of the sterile adapter may provide distinct identification of different tools.

In some embodiments, the movement of the magnets of the sterile adapter may be used to determine whether the tool is properly latched upon attachment. For example, in an embodiment with 3 Hall effect sensors 320 in the IDM, the tool interface plane 712 may be defined based on the position of the three corresponding magnets. Thus, the position of the three magnets in the sterile adapter as sensed by the three Hall effect sensors can be used for determining whether the sterile adapter and/or the tool has been properly latched.

The position of the three magnets corresponding to the tool interface plane 712 of a properly latched tool for a certain type of tool can be hardcoded or stored in the system. When that type of tool is attached, the system can compare the position of the magnets after the tool is attached to the hardcoded or stored position to determine whether the tool is properly latched.

It will be appreciated that while FIGS. 8 and 9 show the plunger contact portions to be generally cylindrical cavities, other shapes are contemplated. For example, the cavity of the plunger contact portion may be designed to receive a conical structure or a pyramidal structure. In some embodiments, the cavity of the plunger contact portion may be designed to receive a structure shaped like a polygonal pillar. In some embodiments, the plunger contact portion may not be a cavity, but rather a protrusion that is designed to move (e.g., by pushing against) the plunger component.

For example, in some embodiments, the plunger contact portion may be a cylindrical or a polygonal pillar designed to depress a free-standing end of a cantilever structure to which a magnet is coupled. In some embodiments, the plunger contact portion may be formed as an integral structure of the housing of the tool.

Some robotic systems detect or determine the type of tool by detecting a magnet provided at the tool using a Hall effect sensor 320 in the IDM. However, one disadvantage of providing a magnet in the tool for the purpose of tool detection using a Hall effect sensor 320 in the IDM is that because the magnet in the tool is separated from the IDM by the sterile adapter in some embodiments, the sensitivity of the Hall effect sensor 320 is relatively low. To compensate for the low selectivity, larger or more magnets need to be used in the tool, resulting in steric hindrance for positioning other tool components such as, for example, the drive couplers and associated pulleys.

The plunger contact portion disclosed herein is constructed as structural component of the tool and enables detection of the tool without including a magnet in the tool. The plunger contact portion disclosed herein enables detection of the tool based on the amount of deflection of the magnet caused by the plunger contact portion when the tool is coupled to the sterile adapter. By providing the plunger contact portion and moving the magnet to the sterile adapter, the need to provide the magnet on the tool is avoided. Moreover more space is available for other tool components, while still providing a method of tool identification using the Hall effect sensor 320 of the IDM.

### F. Method of Coupling a Medical Tool to a Medical Robot

In addition to the various inventive aspects of the medical robotic system, the sterile adapter, and the tool disclosed herein, also described herein is a method of preparing a medical robotic system.

In some embodiments, methods of operating or utilizing the presently disclosed devices and systems may include coupling a sterile adapter to an IDM of the medical robotic system at an IDM interface of the sterile adapter. The sterile adapter is configured to form a sterile barrier between the tool and the IDM. The IDM includes at least one Hall effect sensor disposed adjacent to the IDM interface of the sterile adapter. A tool can then be coupled to the sterile adapter such that a magnet disposed in the sterile adapter is deflected or moved relative to the Hall effect sensor of the IDM.

In some embodiments, the sterile adapter may include a magnet coupled to a plunger component as described herein. In some embodiments, the tool includes a plunger contact portion structured to cause a deflection or movement in the plunger component, and thereby the magnet coupled thereto, relative to the IDM. Such movement may result in changing a magnitude and/or direction of the magnetic field sensed at the Hall effect sensor, which may output a signal based on the detected change. The type of tool being coupled to the sterile adapter (i.e., the medical robotic system) may then be determined based on the signal output by the Hall effect signal.

In some embodiments, the method may further include providing a confirmation or a warning signal based on the identification of the tool being coupled to the robotic system. Such confirmation or warning signal may enable the user, e.g., a physician, of the robotic system to confirm that a tool appropriate for the specific procedure being performed is being coupled to the robotic system.

Similarly, in some embodiments, the method may further include providing a confirmation or a warning signal based on whether the tool is properly latched to the robotic system. Such confirmation or warning signal may prevent the system from driving a non-latched or a partially latched tool, and consequently also prevent improper torque transmission and/or accidental tool detachment.

The method for preparing a medical robotic system disclosed herein facilitates non-visual identification of the tool being coupled to the medical robotic system. In general, when performing a procedure, the user, e.g., a physician, needs to stay focused on the procedure being performed, while monitoring the subject's vital signs, and other medical parameters. Thus, enabling non-visual identification of the tool being coupled to the robotic system improves user comfort and patient safety, thereby resulting in better outcomes for the patients. Advantageously, because the plunger component and the plunger contact portion may be designed to mechanically mate with each other while coupling the tool to the sterile adapter, non-visual alignment of the tool may also be facilitated, thereby further improving user comfort and user attention to the procedure being performed.

### G. Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A sterile adapter for coupling to an IDM of a medical robotic system, the sterile adapter comprising: a housing having a tool interface configured to operatively couple the IDM to a tool such that the sterile adapter transfers torque from the IDM to the tool when the tool is coupled to the sterile adapter; and a magnet coupled to the housing and movable relative to the IDM interface for permitting detection of the tool that is coupled to the sterile adapter. The sterile adapter is configured to form a sterile barrier between the tool interface and the IDM interface.

Clause 2. The sterile adapter of Clause 1, wherein the detection of the tool that is coupled to the sterile adapter comprises detection of at least one of a presence of the tool and a type of tool.

Clause 3. The sterile adapter of any one of the preceding Clauses, wherein the magnet is coupled to a plunger component, the plunger component being configured to move the magnet relative to the IDM interface.

Clause 4. The sterile adapter of Clause 3, wherein the plunger component comprises an elongate body extending parallel relative to a central axis of the sterile adapter, the magnet being disposed in the elongate body.

Clause 5. The sterile adapter of any one of Clauses 3 or 4, wherein the plunger component is configured to move the magnet along an axis oriented parallel relative to a central axis of the sterile adapter.

Clause 6. The sterile adapter of any one of Clauses 3 to 5, wherein the plunger component is configured to move the magnet away from a default position to one of a plurality of sensing positions when the tool is coupled to the sterile adapter.

Clause 7. The sterile adapter of any one of Clauses 3 to 6, wherein the plunger component is configured to move the magnet to one of the plurality of sensing positions based on the type of tool that is coupled to the sterile adapter when the tool is coupled to the sterile adapter.

Clause 8. The sterile adapter of any one of Clauses 3 to 7, wherein the plunger component is biased to a default position using a coil spring.

Clause 9. The sterile adapter of any one of Clauses 3 to 8, wherein the plunger component is biased to a default position using a leaf spring.

Clause 10. The sterile adapter of any one of Clauses 3 to 9, wherein the plunger component comprises an alignment feature for aligning the plunger component relative to the housing during movement of the plunger component.

Clause 11. The sterile adapter of Clause 10, wherein the housing comprises a corresponding alignment feature configured to mate with the alignment feature of the plunger component.

Clause 12. The sterile adapter of any one of Clauses 3 to 11, wherein the adapter comprises two magnets and two plunger components, each magnet being coupled to a respective plunger component.

Clause 13. The sterile adapter of any one of Clauses 3 to 12, wherein the housing defines a tool interface plane and the plunger component comprises a tip portion, and wherein in a default position, the tip portion extends in a direction away from the housing past the tool interface plane.

Clause 14. The sterile adapter of any one of the preceding Clauses, wherein the housing comprises an elastic portion coupled to the magnet, the elastic portion being deformable to permit the magnet to deflect relative to the IDM interface when the tool is coupled to the sterile adapter.

Clause 15. The sterile adapter of any one of the preceding Clauses, wherein the housing comprises rotatably mounted drive members configured to couple with corresponding drive outputs of the IDM and drive inputs of the tool so as to transfer torque from the drive outputs of the IDM drive to the drive input of the tool.

Clause 16. The sterile adapter of Clause 15, wherein the housing comprises five drive members.

Clause 17. The sterile adapter of any one of the preceding Clauses, wherein the magnet is configured to move relative to the IDM interface within a range of about 0.5 mm to about 5 mm.

Clause 21. The sterile adapter of any one of the preceding Clauses, wherein the magnet is movable relative to the IDM interface for permitting detection of at least one of a presence of a tool or a type of tool that is coupled to the sterile adapter.

Clause 22. A robotic surgical system comprising: a medical robot comprising an instrument device manipulator (IDM); a tool to be coupled to the medical robot; and a sterile adapter to be coupled to the IDM for coupling intermediate the tool and the medical robot and forming a sterile barrier between the tool and the IDM, the sterile adapter comprising: a housing having a tool interface configured to operatively couple the IDM to the tool for transferring torque from the IDM to the tool when the tool is coupled to the sterile adapter; and a magnet coupled to the housing and movable relative to the IDM interface for permitting detection of the tool that is coupled to the sterile adapter.

Clause 23. The system of Clause 22, wherein the IDM comprises a Hall effect sensor to sense presence and position of the magnet to permit detection of the type of tool that is coupled to the sterile adapter.

Clause 24. The system of Clause 23, further comprising a controller configured to determine the presence and type of the tool being coupled to the sterile adapter based on a deflection of each of the at least two magnets sensed by a corresponding Hall effect sensor.

Clause 25. The system of any one of Clauses 22 to 24, wherein the system comprises a first tool and a second tool of a different type than the first tool.

Clause 26. The system of Clause 25, wherein the first tool comprises a first adapter interface configured to deflect the magnet to a first sensed position, and wherein the second tool comprises a second adapter interface configured to deflect the magnet to a second sensed position, the first sensed position being different from the second sensed position to permit detection of the type of tool that is coupled to the sterile adapter.

Clause 27. The system of any one of Clauses 22 to 26, wherein the tool comprises an adapter interface having a plunger contact portion configured to cause deflection of the magnet when the tool is coupled to the sterile adapter, wherein the plunger contact portion comprises a cavity in the adapter interface along the housing.

Clause 28. The system of Clause 29, wherein the magnet of the sterile adapter is coupled to a plunger component that is movable relative to the sterile adapter housing, and wherein the plunger contact portion is aligned with and contacts the plunger component when the at least one tool is coupled to the sterile adapter.

Clause 30. The system of Clause 29, wherein the tool comprises a protrusion disposed at a position corresponding to the magnet of the sterile adapter, the protrusion being configured to displace the magnet from a default position relative to the IDM interface.

Clause 31. A tool configured to be coupled to a sterile adapter and an instrument device manipulator (IDM) of a medical robotic system, the tool comprising: a housing having an adapter interface configured to be coupled to a tool interface of the sterile adapter; and a plunger contact portion disposed on the adapter interface and configured to contact and cause deflection of a magnet of the sterile adapter when the surgical tool is coupled to the sterile adapter. An amount of deflection of the magnet of the sterile adapter is configured to enable an identification of the tool.

Clause 32. The tool of Clause 31, wherein the plunger contact portion comprises a cavity in the housing along the adapter interface.

Clause 33. The tool of any one of Clauses 31 to 32, wherein the plunger contact portion comprises a pair of cavities in the housing along the adapter interface, each of the cavities being configured to receive a respective plunger component of the sterile adapter.

Clause 34. The tool of any one of Clauses 31 to 33, wherein the plunger contact portion comprises a protrusion protruding from the adapter interface and configured to cause the deflection of the magnet of the sterile adapter.

Clause 35. A method of preparing a medical robotic system, the method comprising: coupling a sterile adapter to an instrument device manipulator (IDM) of the medical robotic system at an IDM interface of the sterile adapter, the IDM having a Hall effect sensor disposed therein adjacent to the IDM interface of the sterile adapter; and coupling a tool to the sterile adapter to thereby cause movement of a magnet disposed within the sterile adapter relative to the Hall effect sensor of the IDM. The sterile adapter is configured to form a sterile barrier between the tool and the IDM.

Clause 36. The method of Clause 35, wherein the causing movement comprises contacting a plunger contacting portion of the surgical tool against a plunger component of the sterile adapter to cause movement of the magnet.

Clause 37. The method of any one of Clauses 35 to 36, further comprising determining, based on signal output by the Hall effect sensor of the IDM when the surgical tool is coupled to the sterile adapter, at least one of a presence of the tool and a type of the tool that is coupled to the sterile adapter.

Clause 39. A sterile adapter comprising any of the features disclosed herein.

Clause 40. A robotic surgery system comprising any of the features disclosed herein.

Clause 41. A tool comprising any of the features disclosed herein.

Clause 42. A method of operating or manufacturing a tool, sterile adapter, or surgery system comprising any of the features disclosed herein.

### H. Further Considerations

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The singular forms “a,” “an,” and “the” include plural referents unless the context clearly dictates otherwise. Thus, for example, reference to “a plunger component” includes reference to one or more plunger components, and reference to “the magnet” includes reference to one or more magnets.

In one or more aspects, the terms “about,” “substantially,” and “approximately” may provide an industry-accepted tolerance for their corresponding terms and/or relativity between items, such as from less than one percent to five percent.

The term “subject” refers to a mammal that may benefit from the administration using a transdermal device or method of this disclosure. Examples of subjects include humans, and other animals such as horses, pigs, cattle, dogs, cats, rabbits, and aquatic mammals.

As used herein, the term “substantially” refers to the complete or nearly complete extent or degree of an action, characteristic, property, state, structure, item, or result.

It is to be understood that a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.5 to 10 g” should be interpreted to include not only the explicitly recited values of about 0.5 g to about 10.0 g, but also include individual values and sub-ranges within the indicated range. Thus, included in this numerical range are individual values such as 2, 5, and 7, and sub-ranges such as from 2 to 8, 4 to 6, etc. This same principle applies to ranges reciting only one numerical value. Furthermore, such an interpretation should apply regardless of the breadth of the range or the characteristics being described.

Unless otherwise defined, all technical and scientific terms used herein have the same meaning as commonly understood to one of ordinary skill in the art to which this disclosure belongs. Although any methods, devices and materials similar or equivalent to those described herein can be used in the practice or testing of the disclosure, representative methods, devices, and materials are described below.

The word “exemplary” is used herein to mean “serving as an example, instance, or illustration.” Any embodiment described herein as “exemplary” is not necessarily to be construed as preferred or advantageous over some embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes some embodiments not discussed in detail above. Various other modifications, changes and variations may be made in the arrangement, operation and details of the method and apparatus of the subject technology disclosed herein without departing from the scope of the present disclosure. Unless otherwise expressed, reference to an element in the singular is not intended to mean “one and only one” unless explicitly stated, but rather is meant to mean “one or more.” In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

What is claimed is:

1. A sterile adapter for coupling to an instrument device manipulator of a medical robotic system, the sterile adapter comprising:
a housing having a manipulator interface and a tool interface configured to operatively couple the instrument device manipulator to a tool such that the sterile adapter transfers torque from the instrument device manipulator to the tool when the tool is coupled to the sterile adapter; and
a sensor target coupled to the housing and movable relative to the manipulator interface for permitting detection of the tool that is coupled to the sterile adapter,
wherein the sterile adapter is configured to form a sterile barrier between the tool interface and the manipulator interface.

2. The sterile adapter of claim 1, wherein the detection of the tool that is coupled to the sterile adapter comprises detection of at least one of a presence of the tool and a type of tool.

3. The sterile adapter of claim 1, wherein the sensor target is a magnet.

4. The sterile adapter of claim 1, wherein the sensor target is coupled to a plunger component, the plunger component being configured to move the sensor target relative to the manipulator interface.

5. The sterile adapter of claim 4, wherein the plunger component is configured to move the sensor target to one of the plurality of sensing positions based on the type of tool that is coupled to the sterile adapter when the tool is coupled to the sterile adapter.

6. The sterile adapter of claim 4, wherein the plunger component comprises an alignment feature for aligning the plunger component relative to the housing during movement of the plunger component.

7. The sterile adapter of claim 6, wherein the housing comprises a corresponding alignment feature configured to mate with the alignment feature of the plunger component.

8. The sterile adapter of claim 1, wherein the housing comprises an elastic portion coupled to the magnet, the elastic portion being deformable to permit the magnet to deflect relative to the manipulator interface when the tool is coupled to the sterile adapter.

9. A robotic surgical system comprising:
a medical robot comprising an instrument device manipulator and a sensor in the instrument device manipulator;
a tool configured to be coupled to the medical robot; and
an adapter configured to be coupled to the instrument device manipulator intermediate the tool and the medical robot, the adapter comprising:
a housing having a manipulator interface and a tool interface configured to operatively couple the instrument device manipulator to the tool for transferring torque from the instrument device manipulator to the tool when the tool is coupled to the adapter; and
a sensor target coupled to the housing and movable relative to the manipulator interface,
wherein the sensor in the instrument device manipulator is configured to detect movement of the sensor target when the tool is coupled to the adapter.

10. The system of claim 9, wherein the sensor target comprises a magnet and the sensor comprises a Hall effect sensor to sense presence and position of the magnet to permit detection of the type of tool that is coupled to the sterile adapter.

11. The system of claim 10, wherein the adapter is a sterile adapter configured to form a sterile barrier between the tool and the instrument device manipulator.

12. The system of claim 11, wherein the sensor target comprises at least two sensor targets that are independently movable, and wherein the system further comprises a controller configured to determine the presence and type of the tool being coupled to the adapter based on a deflection of each of the at least two sensor targets.

13. The system of claim 9, wherein the tool is a first tool, and wherein the system comprises a second tool of a different type than the first tool, wherein the first tool comprises a first adapter interface configured to deflect the sensor target to a first sensed position, and wherein the second tool comprises a second adapter interface configured to deflect the sensor target to a second sensed position, the first sensed position being different from the second sensed position to permit detection of the type of tool that is coupled to the adapter.

14. The system of claim 9, wherein the tool comprises an adapter interface having a plunger contact portion configured to cause deflection of the sensor target when the tool is coupled to the adapter, wherein the plunger contact portion comprises a cavity in the adapter interface along the housing.

15. The system of claim 14, wherein the sensor target of the adapter is coupled to a plunger component that is movable relative to the adapter housing, and wherein the plunger contact portion is aligned with and contacts the plunger component when the at least one tool is coupled to the adapter.

16. The system of claim 9, wherein the tool comprises a protrusion disposed at a position corresponding to the sensor target of the adapter, the protrusion being configured to displace the sensor target from a default position relative to the manipulator interface.

17. The system of claim 9, wherein the sensor comprises at least one of a Hall effect sensor, a capacitive sensor, or an inductive sensor.

18. The system of claim 9,
wherein the sensor target comprises three sensor targets spaced apart from each other and the sensor comprises three sensors positioned to detect a position of each of a corresponding sensor target,
wherein the system further comprises a controller configured to determine whether the tool is properly latched to the adapter based on a position of the three sensor targets when the tool is coupled to the adapter.

19. A method of preparing a medical robotic system, the method comprising:
coupling a sterile adapter to an instrument device manipulator of the medical robotic system at an manipulator interface of the sterile adapter, the instrument device manipulator having a sensor disposed therein; and
coupling a tool to the sterile adapter to thereby cause movement of a sensor target disposed within the sterile adapter relative to the sensor of the instrument device manipulator,
wherein the sterile adapter is configured to form a sterile barrier between the tool and the instrument device manipulator.

20. The method of claim 19, wherein the causing movement comprises contacting a plunger contacting portion of the surgical tool against a plunger component of the sterile adapter to cause movement of the sensor target.

21. The method of claim 19, further comprising determining, based on a signal output by the sensor of the instrument device manipulator when the surgical tool is coupled to the sterile adapter, at least one of a presence of the tool and a type of the tool that is coupled to the sterile adapter.

22. The method of claim 19, wherein the sensor target comprises three sensor targets and the sensor comprises three sensors positioned to detect a position of each of a corresponding sensor target, wherein the method further comprises determining whether the tool is properly coupled to the adapter based on a position of the three sensor targets when the tool is coupled to the adapter.
